(19) United States
(12) Patent Application Publication
(10) Pub. No.: US 2006/0115804 A1
(43) Pub. Date: Jun. 1, 2006

(54) Method of studying living cells

(76) Inventors: Larry Hench, Bordean Manor (GB); Ioan Notingher, Thornton Heath (GB)

Correspondence Address: FAY, SHARPE, FAGAN, MINNICH & MCKEE, LLP, 1100 SUPERIOR AVENUE, SEVENTH FLOOR, CLEVELAND, OH, 44114, US

(21) Appl. No.: 10/520,913

(22) PCT Filed: Jul. 8, 2003
(86) PCT No.: PCT/GB03/02946

§ 371 Date: Nov. 18, 2005

(30) Foreign Application Priority Data
Jul. 8, 2002 (GB) 02157691

(51) Int. Cl. C12Q 1/00; C12Q 1/68

(52) U.S. Cl. 435/4; 435/6

(57) ABSTRACT

The present invention provides a method of eliciting a Raman signal from a living cell, or a plurality of living cells, said method comprising irradiating the cell with a laser having a wavelength of 785±60 nm.

The present invention relates to a method of studying a living cell or a plurality of living cells. More specifically, the invention relates to a new spectroscopic method of detecting changes that take place in living cells.

### BACKGROUND TO THE INVENTION

Raman spectroscopy has proved to be a versatile technique to study biological samples, providing information regarding molecular structure and interactions and intracellular effects [A. Mahadevan-Jansen, R. Richards-Kortum, Journal of Biomedical Optics 1 (1996), 31-70; G. J. Thomas Jr., Annu. Rev. Biomol. Struct. 28 (1999), 1-27; P. R. Carey, The Journal of Biological Chemistry 274 (1999), 26625-26628; D. Pappas, B. W. Smith, J. D. Winefordner, Talanta 51 (2000), 131-144; D. Naumann, FT-Infrared and FT-Raman Spectroscopy in Biomedical Research, in: Infrared and Raman Spectroscopy of Biological Materials, H. U. Gremlich and B. Yan eds., Marcel Dekker Inc., New York, 2001, pp. 323-377; E. A. Carter, H. G. M. Edwards, Biological Applications of Raman Spectroscopy, in: Infrared and Raman Spectroscopy of Biological Materials, H. U. Gremlich and B. Yan eds., Marcel Dekker Inc., New York, 2001, pp. 421-476; L.-P. Choo-Smith, H. M. Edwards, H. P. Endtz, J. M. Kros, F. Heule, H. Barr, J. S. Robinson Jr., H. A. Bruining, G. J. Puppels, Biopolymers (Biospectroscopy) 67 (2002), 1-9].

Infrared spectroscopy has already been applied to the analysis of cell death and cell cycle [N. Jamin, P. Dumas, J. Moncuit, W.-H. Fridman, J.-L. Teillaud, G. L. Carr, G. P. Williams, Proc. Natl. Acad. Sci. U.S.A 95 (1998), 4837-4840; S. Boydston-White, T. Gopen, S. Houser, J. Bargonetti, M. Diem, Biospectroscopy 5 (1999), 219-227; H. Y. N. Holman, M. C. Martin, E. A. Blakely, K. Bjornstad, W. R. Mckinney, Biopolymers (Biospectroscopy) 57 (2000), 329-335; H. Y. N. Holman, R. Goth-Goldstein, M. C. Martin, M. L. Russel, W. R. Mckinney, Environ. Sci. Technol. 34 (2000), 2513-2517; P. Lash, M. Boese, A. Pacifico, M. Diem, Vibrational Spectroscopy 848 (2002), 1-11] but these studies cannot be carried out in situ due to the strong absorption of water in the infrared region.

Compared to infrared spectroscopy, Raman spectroscopy has the advantage of being a non-invasive technique and biological samples can be studied in their physiological environment due to the low Raman scattering cross-section of water. The high spatial resolution (1 μm) of confocal Raman micro-spectrometers allows measurements of in situ spectra of living cells at different positions inside the cell (e.g. nucleus, cytoplasm) [G. J. Puppels, F. F. de Mul, C. Otto, J. Greve, M. Robert-Nicoud, D. J. Arndt-Jovin, T. M. Jovin, Nature 347 (1990), 301-303; G. J. Puppels, H. S. P. Garritsen, G. M. J. Segers-Nolten, F. F. de Mul, J. Greve, Biophys. J. 60 (1991), 1046-1056; G. J. Puppels, J. Greve, Whole cell studies and tissue characterisation by Raman Spectroscopy, in: Advances in Spectroscopy v25, R. J. H. Clark and R. E. Hester eds., John Wiley & Sons Ltd., Chichester, 1996, pp. 1-47; N. J. Sijtsema, S. D. Wouters, C. J. de Grauw, C. Otto, J. Greve, Appl. Spectrosc. 52 (1998), 348-355; S. Y. Arzhantsev, A. Y. Chikishev, N. I. Koroteev, J. Greve, C. Otto, N. M. Sijtsema, J. Raman Spectrosc. 30 (1999), 205-208].

However, the low Raman scattering efficiency of individual cells makes the measurement difficult. Raman signals can be enhanced by using UV lasers [A. V. Feofanov, A. I. Grichine, L. A. Shitova, T. A. Karmakova, R. I. Yakubovskaya, M. Egret-Chalier, P. Vigny, Biophysical Journal 78 (2000), 499-512] but the strong absorption of nucleic acids and proteins in the UV leads to denaturation which can change the cell phenotype [M. S. Feld, J. R. Kramer, Am. Heart J. 122 (1991), 1803-1805; R. E.-Rasmussen, M. Hammer-Wilson, M. W. Bems, Photochem. Photobiol. 49 (1989), 413-418].

The present invention seeks to provide an improved method of monitoring living cells using Raman spectroscopy which alleviates one or more of the problems associated with prior art techniques. More specifically, the present invention seeks to provide a method of monitoring cells using Raman spectroscopy which minimises cell damage, whilst giving a high signal-to-noise ratio.

### STATEMENT OF INVENTION

In a first aspect, the present invention provides a method of eliciting a Raman signal from a living cell, or a plurality of living cells, said method comprising irradiating the cell with a laser having a wavelength of 785±60 nm.

A second aspect of the invention relates to the use of a laser having a wavelength of 785±60 nm to elicit a Raman signal in a living cell or a plurality of living cells.

### DETAILED DESCRIPTION

As mentioned above, the present invention involves irradiating living cells with a laser having a wavelength of 785±60 nm to elicit a Raman signal. This particular wavelength is capable of exciting strong Raman spectra from living cells over a vibrational range of from about 600 to about 1800 cm⁻¹ without killing the cells.

It is known in the art that in the visible region, although nucleic acids and proteins are expected to have smaller absorptions, even low laser powers can damage the cells. Studies by the applicant have shown that at 488 nm and 514 nm, MLE-12 cells irradiated for short periods of time (for example from 2 to 20 minutes) even at low power levels (of only a few milliwatts, for example 5 mW) exhibit dramatic morphological changes and the Trypan Blue test indicated cell death. Death of the cells was attributed to a photocatalytic process resulting from the absorption of the energetic photons by the DNA and/or proteins molecules. Moreover, at this low level of power, the signal-to-noise ratio in the Raman spectra is poor.

Similar results have been reported by Puppels et al on human lymphocytes [G. J. Puppels, J. H. F. Olminkhof, G. M. J. Segers-Nolten, C. Otto, F. F. de Mul, J. Greve, Experimental Cell Research 195 (1991), 361-367; J. Greve, G. J. Puppels, Raman microspectroscopy of single whole cells, in: Advances in Spectroscopy v20, R. J. H. Clark and R. E. Hester eds., John Wiley & Sons, Chichester, 1993, pp. 231-169]. At the same 488 nm and 514 nm wavelengths, 5 mW irradiation for 5 minutes leads to spectral degradation and the number of living cells is reduced to half. The authors suggest that the cell damage occurrs due to photochemical reactions initiated by the laser [J. Greve et al, ibid].

Thus, to date, photon-induced cell death has precluded the routine use of Raman spectroscopy for in situ analysis of cell metabolism and has made it impossible to follow in real time vital biological processes such as cell mitosis, cell proliferation, cell differentiation, apoptosis (programmed cell death) and cell necrosis.

Studies by the applicant have demonstrated that irradiating living cells with a laser having a wavelength of 785±60 nm leads to Raman spectra with a high signal-to-noise ratio whilst at the same time minimising cell degradation. Advantageously, the living cells can be exposed to this particular wavelength range of laser light for prolonged periods of time (for example, up to 40 minutes or more) without killing the cells. This property enables successive spectra to be measured over a set period of time, thereby allowing changes in the cell to be detected. In particular, the method of the present invention allows individual cells in culture to be sampled over a time period of hours or days, in order to monitor spectral changes that can be correlated with changes in the cell phenotype and cell growth within engineered tissue constructs.

At this wavelength cell degradation and fluorescence are very low and signal strength is reasonably high. Even though at 785 nm Raman scattering is slightly weaker than in the visible region, recent developments of Raman spectrometers with high optical throughput (˜30%) and high sensitivity CCD detectors enable spectra of individual cells to be measured in very short times with reasonable signal-to-noise ratio.

It is notable that there is no teaching or suggestion in the prior art of using a laser having a wavelength of 785±60 nm to elicit Raman signals from living cells.

In a preferred embodiment, the laser used in the presently claimed method has a wavelength of 785±50 nm, preferably 785±40 nm, more preferably 785±30 nm, more preferably still 785±20 nm.

In a particularly preferred embodiment, the cells are irradiated with a laser having a wavelength of 785±10 nm.

In an even more preferred embodiment, the cells are irradiated with a laser having a wavelength of 785±5 nm.

Surprisingly, and in contrast to the teachings of the prior art, using a laser having a wavelength of 785±60 nm to elicit Raman spectra in living cells is associated with a number of unexpected advantages which are outlined below.

In a preferred embodiment, the cell is exposed to a total energy of at least about 20 Joules.

In a more preferred embodiment, the cell is exposed to a total energy of at least about 50 Joules.

In an even more preferred embodiment, the cell is exposed to a total energy of at least about 100 Joules, more preferably at least about 150 Joules, and more preferably still at least about 200 Joules.

Even more preferably, the cell is exposed to a total energy of at least 250 Joules, and more preferably at least 275 Joules.

It is notable that there is no teaching or suggestion in the prior art that using a laser having a wavelength of 785±60 nm to elicit Raman signals from living cells would lead to the unexpected advantages in terms of the energy which the cells can tolerate without exhibiting the signs of significant spectral degradation.

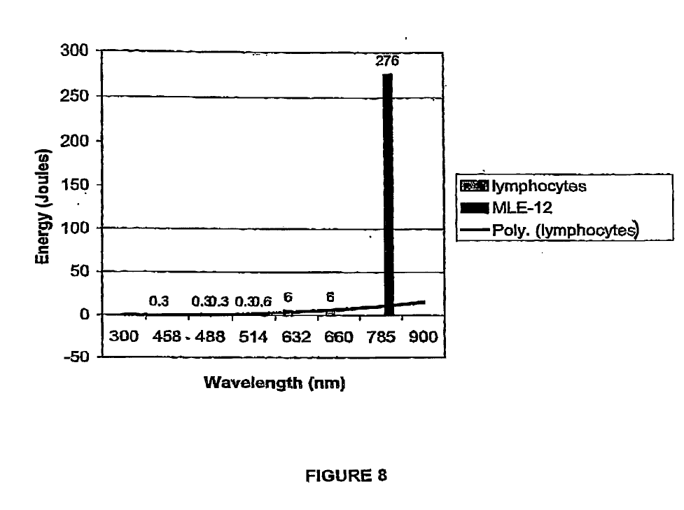

Indeed, extrapolation based on experiments irradiating cells at wavelengths between 457.5 and 660 nm suggests that the maximum laser energy to maintain cell viability at 785 nm should be around 10 Joules. In contrast, the actual maximum laser energy to maintain cell viability at 785 nm was found to be 276 Joules, i.e. over 25-fold higher than anticipated. This effect is further illustrated in FIG. 8.

In one preferred embodiment of the invention, the cell is irradiated at an intensity of 115±50 mW.

In one especially preferred embodiment, the cell is irradiated with a laser having a wavelength of 785±20 nm at an intensity of 115±50 mW. Advantageously, irradiating the cells with a laser of 785±20 nm at an intensity of 115±50 mW produces Raman spectra with an excellent signal-to-noise ratio but which does not kill the cells.

In an especially preferred embodiment, the cell is irradiated with a laser having a wavelength of 785±20 nm at an intensity of 115±50 mW for a period of up to 40 minutes.

In another particularly preferred embodiment, the cell is irradiated for a period of up to 90 minutes at a laser power of 115±50 mW.

In an alternative preferred embodiment, the cell is irradiated at an intensity of 120±60 mW.

In another preferred embodiment, the cell is irradiated with a laser having a wavelength of 785±60 nm at an intensity of 120±60 mW.

In an especially preferred embodiment, the cell is irradiated with a laser having a wavelength of 785±60 nm at an intensity of 120±60 mW for a period of up to 40 minutes.

In a particularly preferred embodiment, the cell is irradiated for a period of up to 90 minutes at a laser power of 120±60 mW.

In one preferred embodiment of the invention, the laser is focussed within the cytoplasm of the cell. Thus, 785±60 nm laser light can be focused inside the cytoplasm to elicit Raman spectra characteristic of a wide variety of intracellular proteins without killing the cell.

In another preferred embodiment, the laser is focussed within the nucleus of the cell. Thus, 785±60 nm laser light can be focused to a spot within the nucleus of a living cell in situ and excite a Raman spectrum characteristic of the nucleic proteins and DNA in the nucleus, including specific peaks assigned to each of the nucleotides (A, T, G, C) as well as spectral peaks characteristic of the DNA backbone and conformational folding without damaging the genetic material of the cell.

In yet another preferred embodiment, the laser is focussed within the extracellular matrix. Thus, focussing the 785±60 nm laser outside the cell elicits the Raman spectrum of the extracellular matrix, including inorganic phases, such as hydroxyapatite.

In one preferred embodiment, the laser can be use to elicit Raman signals from a cluster of living cells (for example, n≈5) to produce a spectrum similar to individual cells, without inducing cell death. Thus, multiple cells can be studied at the same time, for example in order to monitor the behaviour of cells growing into a tissue construct, or to monitor the effects of toxic agents or pharmaceutical agents on an assemblage of living cells in the presence of their extracellular matrix.

The method of the present invention may be used to monitor changes in a living cell, or a plurality thereof. Preferably, the cell is cultured on a bioinert material.

Even more preferably, the bioinert material is poly-L-lysine coated fused silica.

In one especially preferred embodiment, the bioinert material is magnesium fluoride (MgF₂) or fused silica (SiO₂).

In one preferred embodiment, the cell is cultured on a bioactive scaffold.

In another preferred embodiment, the cell is cultured on an uncoated bioactive glass or a sol-gel derived gel glass. One example of such an uncoated bioactive glass is 45S5 Bioglass®. An example of such an uncoated gel glass is mesoporous silica (SiO₂) gel glass. A further example of such an uncoated gel glass is 70 mol % SiO₂, 30 mol % CaO.

Another preferred embodiment of the invention relates to a method of detecting changes in a living cell or a plurality of living cells, said method comprising the steps of:
(i) eliciting a Raman signal in accordance with the method of the invention as described above; and
(ii) measuring changes in the Raman signal over a period of time.

In one particularly preferred embodiment, the method is used for detecting changes in the cell phenotype.

In another particularly preferred embodiment, the method is used for monitoring cell growth, including cell division (mitosis).

In yet another preferred embodiment, the method is used for detecting changes in a living cell induced by a pharmaceutical agent or a cytotoxic agent.

In another preferred embodiment, the method is used for detecting changes in the cell cycle.

In one especially preferred embodiment, the method is used for detecting changes in protein levels.

In another especially preferred embodiment, the method is used for detecting changes in DNA or RNA levels.

In yet another preferred embodiment, the method is used for detecting changes in the extracellular matrix.

In view of the lack of completely non-invasive real-time techniques to monitor the biological events at a cellular level, the present invention represents an important breakthrough in the fields of cell biology and tissue engineering. The potential applications of the present technology are numerous, ranging from the study of intrinsic mechanisms within cells to the interaction of cells with external factors.

By way of example, the study of cell proliferation, cell differentiation and cell death is now possible without killing the cells. The response of pathological and cancerous cells to different chemical factors can also be tested, for example, in photodynamic therapy and chemotherapy. Circumventing photon induced cell death during in situ bio-optical spectral analysis is especially important because of the potential for non-invasive in vivo monitoring of pre-cancerous lesions [Mahadevan-Jansen et al, ibid; T. C. Bakker, G. J. Puppels, Y. M. Kraan, J. Greve, L. L. J. Van Der Maas, C. G. Figdor, Int. J. Cancer (Pred. Oncol) 74 (1997), 20-25; S. R. Hawi, W. B. Campbell, A. Kajdacsy-Balla, R. Murphy, F. Adar, K. Nithipatikom, Cancer. Letters 110 (1996), 35-40] and tissue pathologies [E. B. Hanlon, R. Manoharan, T.-W. Koo, K. E. Shafer, J. T. Motz, M. Fitzmaurice, J. R. Kramer, I. Itzkan, R. R. Dasari, M. S. Feld, Phys. Med. Biol. 45 (2000), R1-R59; R. Wolthuis, T. C. Bakker Schut, P. J. Caspers, H. P. J. Bushman, T. J. Romer, H. A. Bruining, G. J. Puppels, Raman Spectroscopic Methods for In Vitro and In Vivo Tissue Chararacterisation, in: Fluorescent and Luminescent Probes for Biological Activity, W. T. Mason ed., Academic Press, London, 1999, pp. 433-455; L.-P. Choo-Smith et al, ibid].

Further applications lie in the field of tissue engineering, where cell attachment to different bioactive 3D scaffolds and cellular interactions with released ions can be studied. In this regard, it is important to be able to monitor live cellular behaviour in vitro as a means of obtaining feedback control of bioreactors for tissue engineering and non-invasive functional analysis of tissue engineered constructs, pre and post implantation in a patient.

The in situ monitoring of cell interactions with various chemical substances is also of importance. In particular, the in situ optical monitoring of living cells will make it possible to study cells in biodetector systems for toxic compounds and for the testing of pharmaceuticals. For example, in situ analysis of changes in protein synthesis, DNA or RNA at various dosages of a candidate drug can be studied prior to animal trials. With regard to bio-optical detection systems for toxic agents, cellular tolerance to long irradiation times allows for the continuous sampling of live cells in combination with real-time software analysis for the identification and quantification of toxic agents.

The invention is further described by way of example and with reference to the following figures wherein:

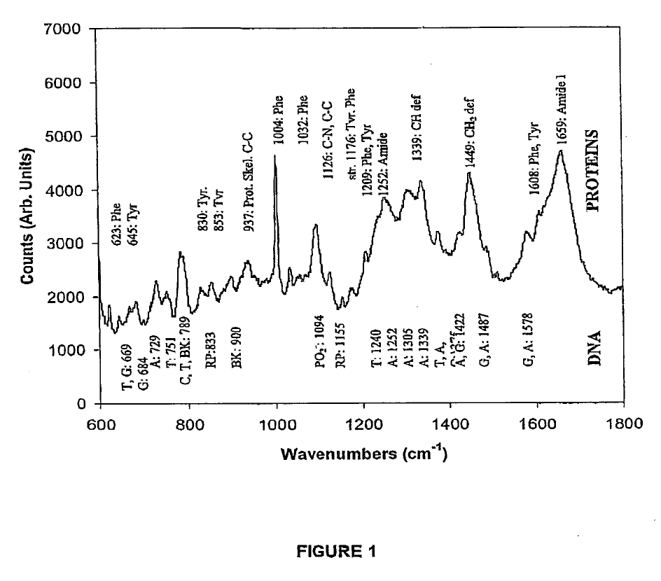

FIG. 1 shows a Raman spectrum of a cluster of cultured lung cells. Spectral assignment: Lower part: DNA (A, G, T, C: adenine, guanine, thymine, cytosine), BK: backbone, RP: ribose-phosphate). Upper part: Proteins (Phe: phenylalanine, Tyr: Tyrosine).

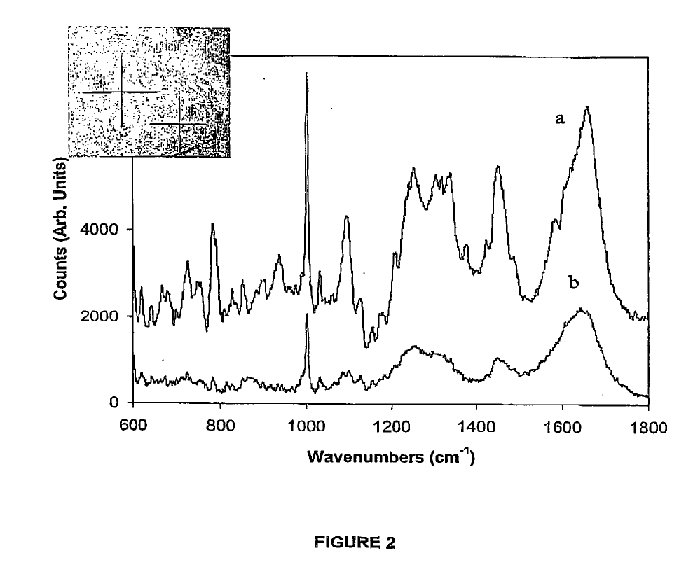

FIG. 2 shows Raman spectra of an individual cell; a) nucleus, b) cytoplasm.

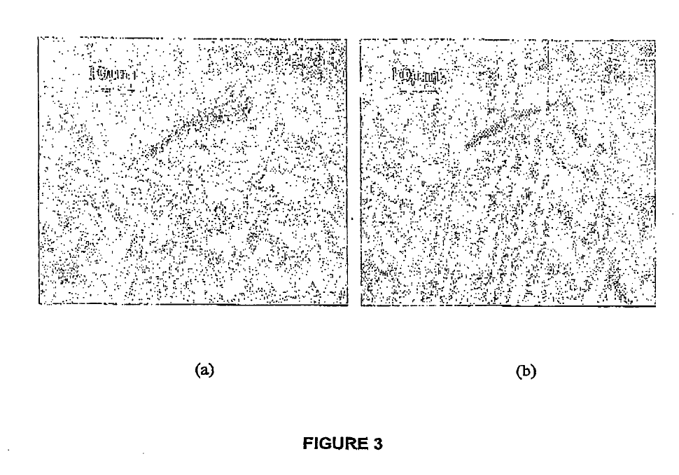

FIG. 3 shows pictures of MLE-12 cells before (a) and after 40 minutes irradiation with 115 mW 785 nm laser (b). The cell in picture (b) did not colour blue after treatment with Trypan Blue

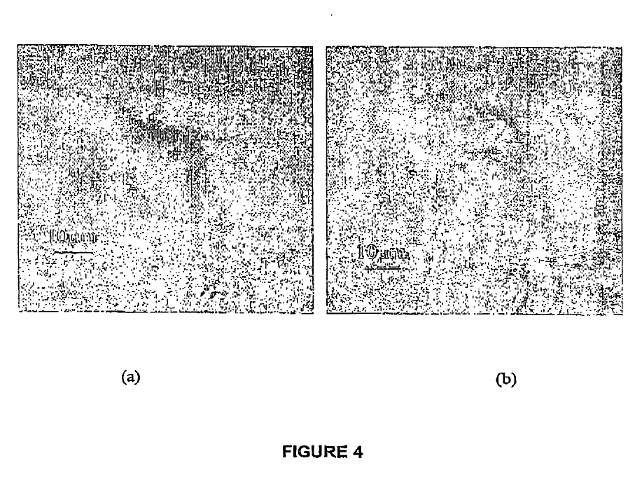

FIG. 4 shows the effect of laser irradiation at 488 nm on MLE-12 cells. a) initial, b) after 10 minute irradiation at 5 mW laser power.

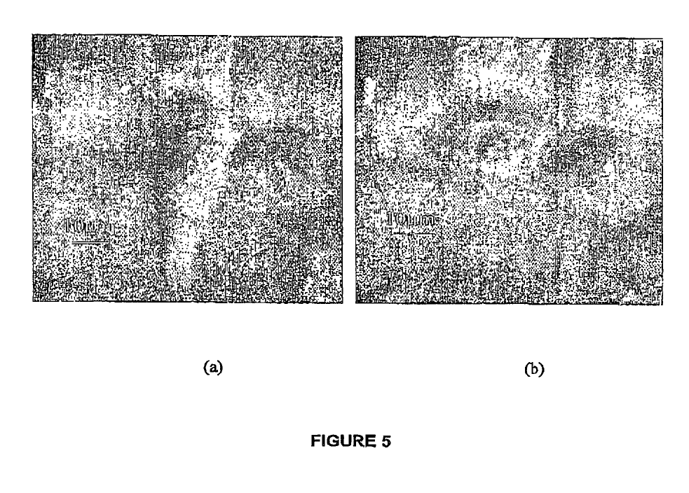

FIG. 5 shows the effect of laser irradiation at 514 nm on MLE-12 cells. a) initial b) after 20 minute irradiation at 5 mW laser power.

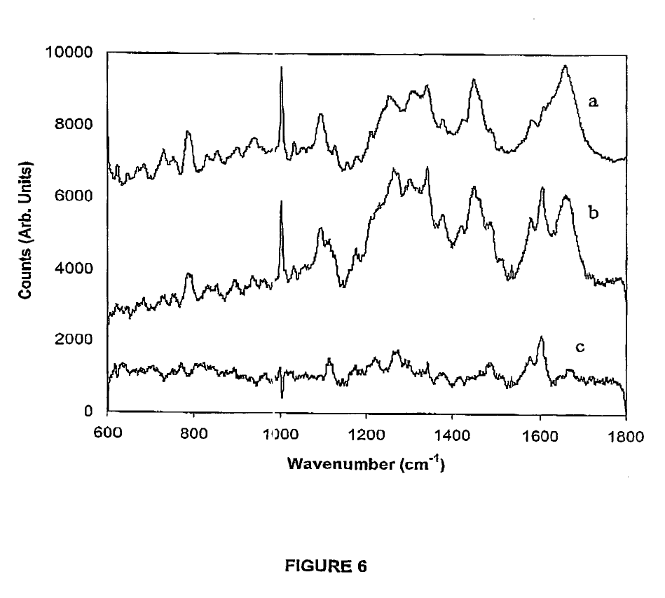

FIG. 6 shows Raman spectra of individual living (a) and dead (b) MLE-12 cells; (c) calculated difference spectrum (b)−(a). The most important differences between the spectra of living and dead cells are in the 1530-1700 cm⁻¹ range, where the dead cells have strong peaks at 1578 cm⁻¹ and 1607 cm⁻¹. Other differences occur around the DNA peak at 1095 cm⁻¹.

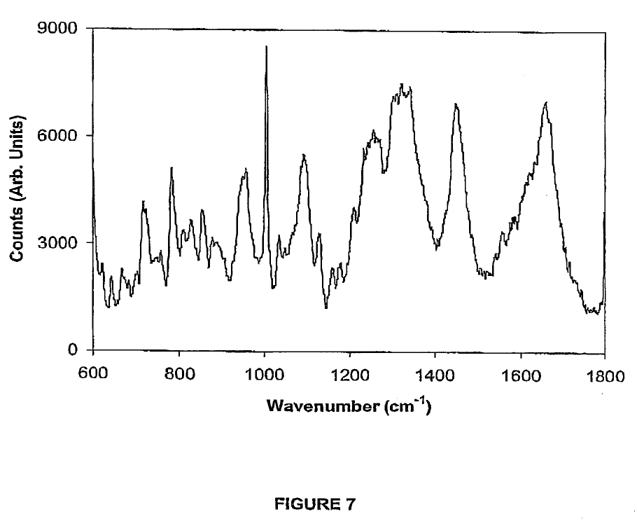

FIG. 7 shows the Raman spectrum of an individual lung cell on 45S5 Bioglass®. The peak at 960 cm⁻¹ corresponds to hydroxyapatite (HA).

FIG. 8 shows the maximum laser energy to maintain 100% cell viability for MLE-12 cells irradiated at 488, 514 and 785 nm, compared to the maximum laser energy for lymphocytes irradiated at 457.9, 488, 514, 632 and 660 nm [Puppels et al, Exp. Cell Res. 1991]. The solid black line shows that the predicted maximum laser energy to maintain 100% cell viability at 785 nm (based on extrapolation from the data points for lymphocytes and MLE-12 cells irradiated at wavelengths between 457.5 and 660 nm) is around 10 Joules. In contrast, the actual maximum laser energy to maintain 100% cell viability at 785 nm was found to be 276 Joules.

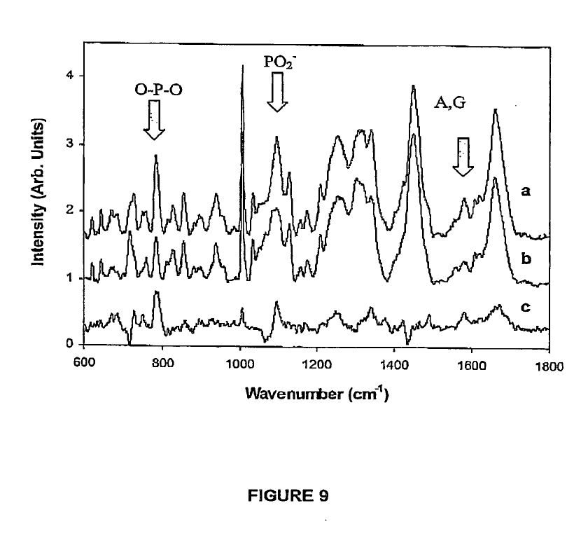

FIG. 9 shows the Raman spectra of round A549 cells in the upper layer (a), surrounded cells in the continuous layer (b) and the computed difference spectrum (c) (Arrows indicate positions of most significant variations).

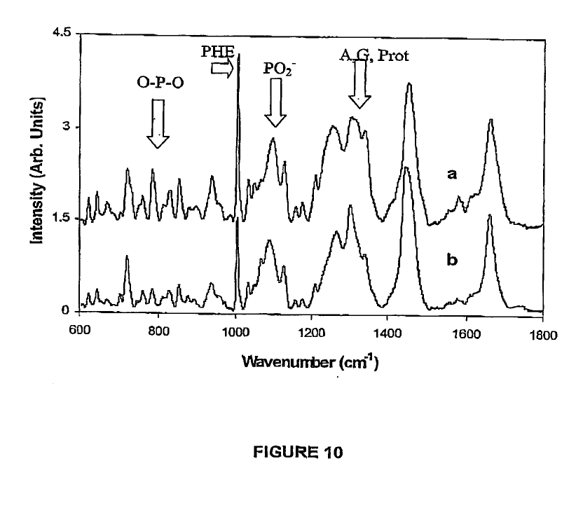

FIG. 10 shows the Raman spectrum of viable (a) and dead (b) A549 cells (Arrows indicate the positions where most significant changes occur).

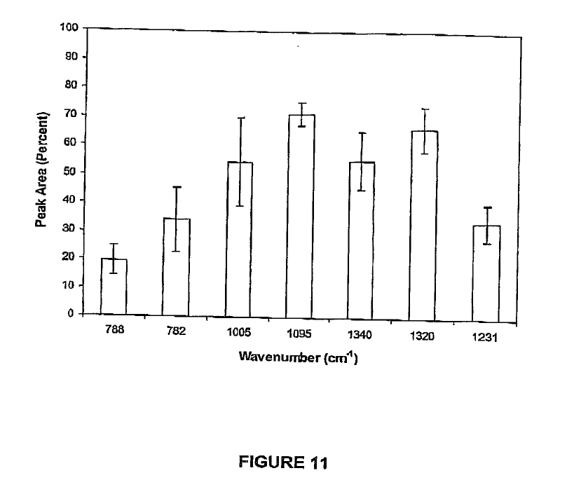

FIG. 11 shows the decrease in DNA and protein peaks in the Raman spectra of dead A549 cells.

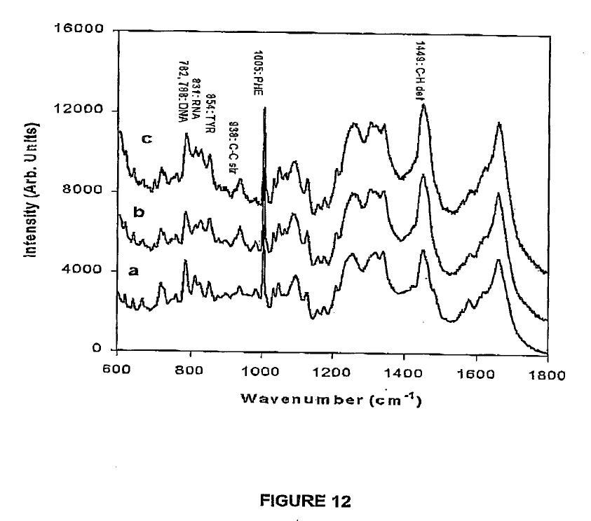

FIG. 12 shows the Raman spectra of undifferentiated murine stem cells (a) and after 16 (b) and 20 (c) days of differentiation.

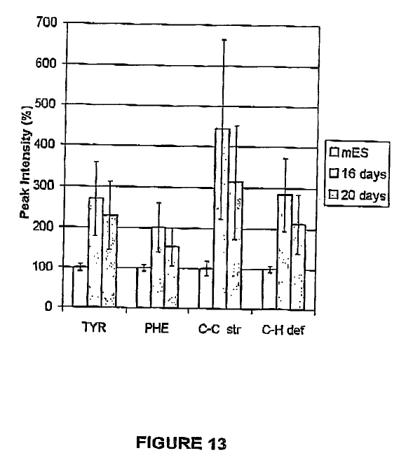

FIG. 13 shows the increase in the protein content in mES cells during differentiation.

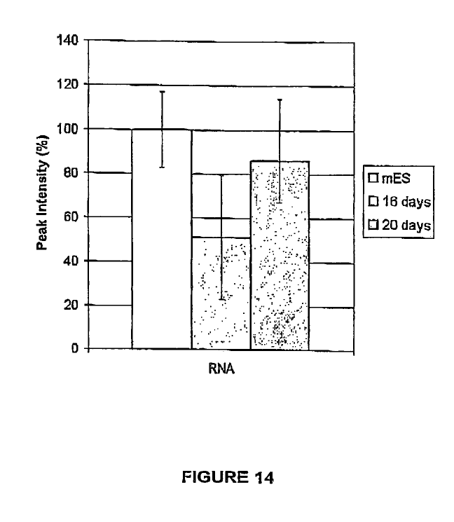

FIG. 14 shows the decrease in the RNA content in mES cells during differentiation.

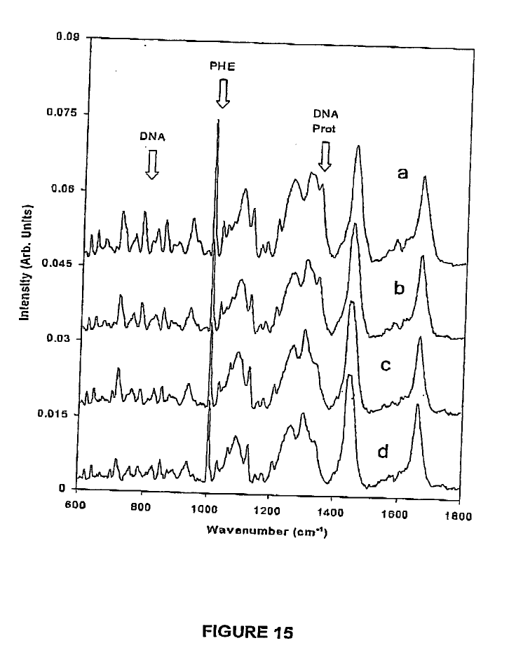

FIG. 15 shows the Raman spectra of viable A549 cells (a) and after 24 (b), 48 (c) and 72 (d) hours of Triton X100 treatment.

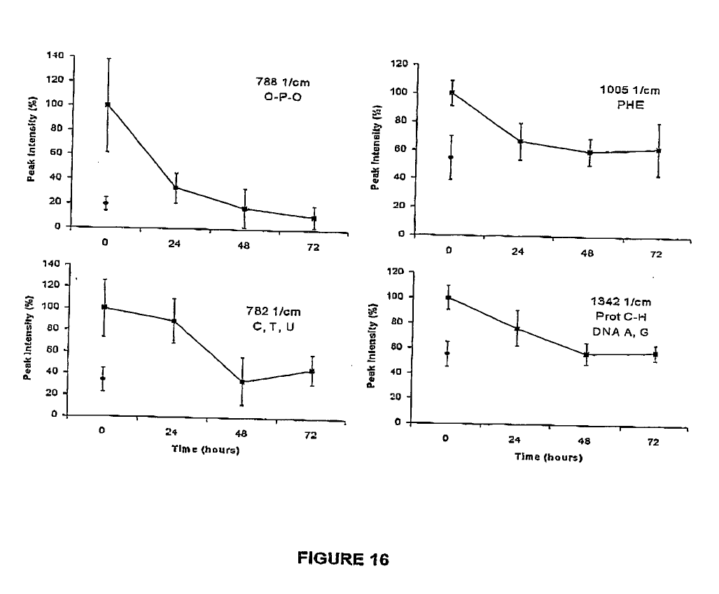

FIG. 16 shows the decrease in the Raman peaks of DNA (a) and proteins (b) following treatment with Triton X100 (▪ measured values, ● values corresponding to dead cells in FIG. 10).

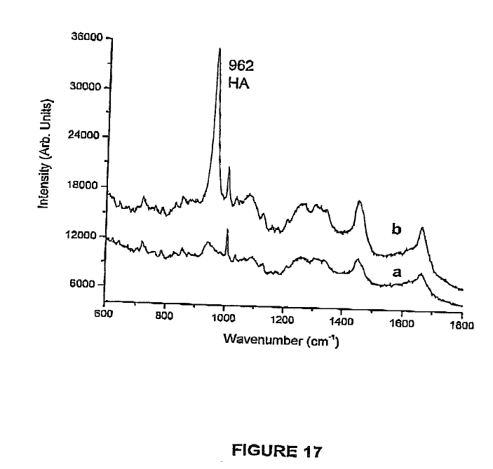

FIG. 17 shows the Raman spectra of primary human osteoblasts cultured on 45S5 Bioglass®: (a) without BGP, (b) with BGP.

### EXAMPLES

Sample Preparation

MLE-12 cells from passage 6 to 15 were used. These cells are immortalised murine lung epithelial cells [K. A. Wikenheiser, D. K. Vorbroker, W. R. Rice, J. C. Clark, C. J. Bachurski, H. K. Oie, J. A. Whitsett, Proc Natl Acad Sci USA. 90 (1993), 11029-11033] that conserve a differentiated phenotype until at least the 30^{th} passage. The cells were seeded at a fixed density (2.10⁴ cells/cm²) on 45S5 Bioglass® discs and poly-L-lysine (30-70kDa, 30 μg/ml) coated fused silica substrates and incubated in HITES culture medium at 37° C. 5% CO₂ for 24 hours [K. A. Wikenheiser et al, ibid). Before analysis, samples were rinsed and immersed in standard PBS solution. For the measurement of Raman spectra of dead cells, cells were prepared in the same conditions and left in the incubator for 4 days without changing the culture medium in order to ensure a high percentage of dead cells. For cell viability tests, a standard Trypan Blue dye method was used after the Raman measurements were completed [D. C. Allison, P. Ridolpho, J. Histochem. Cytochem. 28 (1980), 700-703]. This test relies on the alteration in membrane integrity as determined by the uptake of dye by dead cells.

Measurement of Raman Spectra

The spectra were measured with a Renishaw 2000 Raman micro-spectrometer equipped with a 785 nm diode laser for excitation. A 63× magnification 0.90 numerical aperture water immersion Leica objective was used with a free working distance of 2 mm to insure minimal invasion to the cells. For measurements, the laser power was 115 mW and the signal was integrated for 120 sec. After collection, the spectra were corrected against the influence of the substrate and medium and a quintic function was used for baseline correction. To study the effect of visible lasers on the cells, the 785 nm laser was replaced with an Argon ion laser at 488 nm and 514 nm wavelength. At these wavelengths, the power used was only 5 mW.

Results

A typical Raman spectrum for a cluster (approximately 5 cells) of MLE-12 cells cultured on poly-L-lysine coated fused silica is presented in FIG. 1. Table I summarises the spectral peak assignments shown in FIG. 1. Peak assignments are based on references [A. Mahadevan-Jansen et al, ibid, D. Naumann et al, ibid, E. A. Carter et al, ibid, Puppels et al, Biophys. J. 1991, ibid; S. A. Overman, K. L. Aubrey, K. E. Reilly, O. Osman, S. J. Hayes, P. Serwer, G. J. Thomas Jr., Biospectroscopy 4 (1998), S47-S56]. The Raman spectrum of the living MLE-12 cells is dominated by vibration bands of the nucleic acids and proteins, the contribution from the membrane lipids being negligible. The Amide I band centred at 1659 cm⁻¹ together with the position of the C—C skeletal vibrations at 937 cm⁻¹ suggest that the predominant conformation of the proteins in the MLE-12 cells is a-helical [6]. The DNA bands at 1094 cm⁻¹ and 833 cm⁻¹ indicate that the DNA is in the B form [Puppels et al, Biophys J, 1991, ibid].

The high spatial resolution of the Raman micro-spectrometer allows spectra to be collected from different positions inside the same cell. The spectral difference between the nucleus and cytoplasm for a single living lung cell is illustrated in FIG. 2. The spectrum corresponding to the cytoplasm is much weaker and as expected lacks the peaks associated with DNA. This suggests that the nuclei have the strongest contribution to the spectrum of the cell cluster presented in FIG. 1. Only small variations occurred between the spectra measured of different cells (n=10) and at different positions (n=3) in the nucleus. These differences correspond mainly to the amount of nucleic bases, adenine, guanine, thymine and cytosine at 1487 cm⁻¹, 1422 cm⁻¹; 1375 cm⁻¹ and 789 cm⁻¹. The standard deviation of the integrated Raman signal between 1150 cm⁻¹ and 1510 cm⁻¹ is about 5%. However the protein to DNA ratio calculated as the ratio of the areas corresponding to the peaks at 1449 and 1095 cm⁻¹ indicated variations in the range 1.9-2.7 depending on the focusing position in the nucleus. These values are very similar to the value reported for granulocytes using Raman spectroscopy [Puppels et al, Biophys J, 1991, ibid].

In order to understand the effect of the 785 nm laser on the MLE-12 cell, three short measurements of only 80 sec were taken during a 40 minute laser irradiation at the same laser wavelength (785 nm) and power (115 mW) as used for measurements. FIG. 3 show micrographs of a typical MLE-12 cell before (FIG. 3a) and after (FIG. 3b) the 40 minutes of irradiation. The cell in FIG. 3b changed slightly in shape and its spectrum did not change; the absence of Trypan Blue staining proved the cell viability. When the same experiment was repeated using 488 nm and 514 nm lasers at 5 mW laser power, the morphology of the cells changed dramatically after times as short as 5 minutes. The cells coloured blue by the Trypan Blue test, indicating that the cells were dead. Micrographs of these cells before and after visible laser irradiation are shown in FIGS. 4 (488 nm, 5 mW for 10 minutes) and 5 (514 nm, 5 mW for 20 minutes).

To demonstrate the capability of Raman spectroscopy to distinguish between living cells and dead cells, the spectra of cells deliberately left in the incubator for 4 days without changing the culture medium were measured. A Trypan Blue test, revealed a high proportion of dead cells. To ensure that the measured cells were dead, and in order to avoid any interference to the measured spectrum, the Trypan Blue test was carried out after each Raman measurement. A typical spectrum of a dead cell is shown in FIG. 6a, together with the spectrum of a living cell for comparison (FIG. 6b). Cell death produces numerous changes in both protein and DNA spectral features, as indicated in the difference spectrum (FIG. 6c). The main differences are in the 1530-1700 cm⁻¹ and 1070-1150 cm⁻¹ spectral ranges. The dead cell spectrum has high peaks at 1578 and 1607 cm⁻¹ and also a new peak at 1114 cm⁻¹.

For tissue engineering applications, the cells must be able to attach, proliferate and maintain a differentiated phenotype for long periods of time. Poly-L-lysine coated fused silica is a bioinert material, therefore, the objective is oriented towards bioactive scaffolds made of uncoated sol-gel derived bioactive glasses and gel-glasses [W. Cao, L. L. Hench, Ceramics International 22 (1995), 493-507; L. L. Hench, J. K. West, Life Chemistry Reports 13 (1996), 187-241; L. L. Hench, Biomaterials 19 (1998), 1419-1423; J. R. Jones, L. L. Hench, Materials Science and Technology 17 (2001), 891-900]. It has been previously shown that 45S5 Bioglass® (45% SiO₂, 24.5% Na₂O, 24.5% CaO and 6% P₂O₅ in weigth) has an active role in cell adhesion and proliferation related to gene activation caused by cell-substrate interaction (e.g. controlled ion release) [I. D. Xynos, M. V. Hukkanen, J. J. Batten, L. D. Buttery, L. L. Hench, J. M. Polak, Calcif. Tissue Int. 67 (2000), 321-329; I. D. Xynos, A. J. Edgar, L. D. Buttery, L. L. Hench, J. M. Polak, Biochemical and Byophysical Research Communications 276 (2000), 461-465]. However, the Raman spectrum of 45S5 Bioglass® lacks the simplicity of fused silica with a stronger signal due the P—O vibrations, making the measurements and data processing more difficult. The Raman spectrum of an individual MLE-12 lung cell from cells grown on a 45S5 Bioglass® disc is shown in FIG. 7. The main difficulty in obtaining the Raman spectrum of the cell is that subtraction of the 45S5 Bioglass® signal around 960 cm⁻¹ leads to an error due to the strong band corresponding to the symmetric stretching of the P—O groups of hydroxy apatite (HA) which forms at the bioactive surface [I. Rehman, L. L. Hench, W. Bonfield, Bioceramics 6 (1993), 123-128]. However, this distortion does not interfere with the aim of the present invention because apart from covering the skeletal C—C vibration of proteins at 937 cm⁻¹, which makes the determination of the protein secondary structure a bit more difficult, no interference with the DNA bands occurs.

There are some differences between the spectrum in FIG. 7 compared to those in FIGS. 1 and 2, especially the 1487 cm⁻¹, 1422 cm⁻¹, 1375 cm⁻¹ and 1252 cm⁻¹ bands which are due to the DNA bases. These small variations have also been observed when poly-L-lysine coated fused silica was used as substrate and various cells were measured. However, spectral location of the bands regarded as markers for DNA and protein secondary structure are equivalent in FIGS. 1, 2 and 7, which suggest that the 45S5 Bioglass® substrate does not disturb the DNA or the conformations of proteins within the cell.

Following the unexpected finding that the use of a high-power 785 nm laser (≈120 mW at sample) does not induce cell death to living cells even after one hour of continuous exposure, studies were undertaken to investigate the significance of the measured Raman spectra of individual cells. Firstly, studies correlated Raman spectral changes with biochemical and biophysical changes within living cells during fundamental biological processes, such as progression through cell cycle, cell death and differentiation. Secondly, changes in cell behaviour were monitored when the cells were exposed to chemicals, various substrates, toxins or drugs. The results of these experiments are described below.

(i) Cell Cycle

Since peaks corresponding to DNA vibrations (both DNA strands and nitrogenous bases) are present in the Raman spectra of living cells, the magnitude of these peaks should change as cells progress through the cell cycle. In order to see the differences between the cells in different cell cycle phase, cells were seeded in a higher density so that they formed a continuous cell layer, with round cells on top. As each cell in the continuous layer is surrounded by other cells and has less access to nutrients, it is likely to be in the G1/G0 phase. In contrast, the round cells in the superior layer have enough space around them to divide and therefore can be in any stage of the cycle, including cell mitosis (S/G2 phase).

The differences between Raman spectra of round cells and cells within the continuous layer are shown in FIG. 9. The round cells show a higher amount of nucleic acid as indicated by the higher peaks at 788 cm⁻¹ (100% increase), 1095 cm⁻¹ and 1578 cm⁻¹. These differences confirm the hypothesis that the cells in the lower layer are in G1/G0 phase and have a smaller amount of nucleic acid compared to the round cells in the upper layer, which are likely to be undergoing cell mitosis in S/G2 phase.

(ii) Cell Death

Cell death can occur due to various factors and involves many biochemical and biophysical changes in the cell. In particular, cell death is associated with denaturation and conformational changes of proteins as well as fragmentation of DNA. Studies investigated dying cells in fresh culture medium, since a culture always contains a small percentage of dead cells. A small number of cells showing fragile cell attachment were visually identified and their Raman spectrum measured. After the Raman spectra were measured, a Trypan blue viability test was carried out to confirm cell death. FIG. 10 shows the Raman spectra of living and dead A549 cells.

The Raman spectra of these cells showed large spectral differences compared to the well attached, stable cells. The spectrum of dead cells show a large decrease in intensity of the peaks corresponding to nucleic acids at 786 cm⁻¹ and 1095 cm⁻¹ and also a big decrease in the peak corresponding to phenylalanine at 1005 cm⁻¹.

To quantify the spectral differences between viable and dead A549 cells (which could be used in further studies as markers of cell death), peak fitting was carried out in the spectral regions where the most dramatic changes were observed: 770-803 cm⁻¹, 995-1020 cm⁻¹, 1020-1150 cm⁻¹, 1190-1385 cm⁻¹. The results of this analysis are shown in FIG. 11 as percentage relative to the values in viable A549 cells.

The molecular vibration that is most sensitive to cell death seems to be the O—P—O stretching of the DNA at 788 cm⁻¹ which decreases by 80%. This dramatic decrease indicates that breakage of phoshodiester bonds lead to fragmentation of the DNA strands. Besides the decrease of the peaks corresponding to nucleic acids, the Raman spectrum of dead A549 cells indicates changes in protein vibrations. FIG. 11 shows that there is a strong decrease by 45% of the 1005 cm⁻¹ peak corresponding to phenylalanine. A change in the protein conformation can also be observed. The reduction by 66% of the protein peak at 1231 cm⁻¹ corresponding to random coils indicates that the protein strands become associated more in α-helixes and β-sheets during cell death. This is also confirmed by the reduction in the width of the 1660 cm⁻¹ Amide I band from 38 cm⁻¹ to 28 cm⁻¹. A similar change in the protein conformation in dead cells was previously reported in a study of cell death using synchrotron FTIR microscopy.

(iii) Cell Differentiation

Murine embryonic stem (mES) cell lines are pluripotent cells, derived from early embryo that can be propagated and induced into differentiation in vitro. The mES were induced into differentiation via formation of embryoid bodies. Raman spectra were measured after 16 and 20 days of differentiation (FIG. 12). The spectra were normalised to the DNA peak at 788 cm⁻¹ assuming that the amount of DNA is stable during the differentiation process.

FIG. 13 presents the magnitude of the peaks corresponding to proteins for undifferentiated ES cells and differentiated cells via embryoid body after 16 and 20 days. The most significant change occurs at 938 cm⁻¹, where an increase by 343% is observed after 16 days of differentiation (P=0.0024) and 212% after 20 days (P=0.0006). Also significant increase of the C—H peak is observed: 184% (P=0.0001) at 16 days and 112% (P=0.0005) at 20 days in culture. The magnitudes of the peaks corresponding to amino acids are also significantly higher: 169% after 16 days (P=0.0007) and 128% after 20 days (P=0.0005) for the tyrosine, and 100% (P=0.0008) after 16 days and 57% after 20 days (P=0.0047) for phenylalanine.

The significant increase of protein in differentiated cells corresponds to the increased production of specific proteins commonly observed during cell differentiation. The variations observed in the Raman spectra of differentiated cells could be due to the fact that the culture is a mix of cells with different phenotypes, as observed with the different cell morphologies.

The concentration of single strand RNA is proportional to the peak at 813 cm⁻¹ in the Raman spectrum of living cells. Variations in the amount of RNA in the mES cells during differentiation are shown in FIG. 14. The intensity of RNA peak decrease by 50% for the differentiated cells after 16 days in culture (P=0.0044). An increase in the RNA concentration after 16 days is suggested by the increase in the RNA peaks in the Raman spectra corresponding to cells after 20 days of differentiation.

The high concentration of RNA observed in mES cells suggests that RNA is not translated. This agrees with the low amount of protein observed. The decrease of RNA in the differentiated cells can be correlated with the increase of the protein quantity observed in FIG. 13. The cells appear to use the pool of RNA observed in ES cells to produce new specific protein, during the differentiation. These results agree with previous reports in literature, where non-translated mRNA (mRNA in repression) was showed in early embryogenesis.

(iv) Effect of the Toxic Detergent Triton x100 on A549 Cells

The Raman spectra of A549 cells exposed to 100 μM Triton X100 after 24, 48 and 72 hours are shown in FIG. 15.

Comparing FIG. 15 and FIG. 10, one can see that following the Triton X100 treatment, the corresponding Raman spectra undergo changes similar to the spectra of dying cells. FIG. 16 shows the decrease in the DNA and protein Raman peaks as function of treatment time.

(v) Formation and Mineralisation of Bone Nodules

Primary human osteoblasts were cultured on 45S5 Bioglass® substrates in order to induce formation of new bone. The formation of new bone can be detected with high sensitivity using Raman spectroscopy with 785 nm lasers by detecting the 962 cm⁻¹ Raman peak of hydroxyapatite (the mineral phase of bone). However, after 10 days in culture, no evidence of formation of bone nodules was observed in the Raman spectra (FIG. 17). Only peaks corresponding to intrinsic cellular components were measured.

However, when the culture medium of the bone cells was supplemented with beta-glycerophosphate (BGP), a strong peak corresponding to hydroxyapatite was detected in the Raman spectra (FIG. 17).

By way of summary, the present invention demonstrates that confocal Raman micro-spectroscopy is suitable for the in situ characterisation of individual living cells cultured on inert silica and bioactive sol-gel derived glass (45S5 Bioglass®). We have shown that at 785 nm wavelength, laser powers as high as 115 mW or 120 mW can be used for times as long as 40 minutes or more to monitor continuously the biological state of the cells without altering the cell spectra and morphology or inducing cell death. Substantial differences between the spectra of living and dead MLE-12 cells have been established. Studies have demonstrated that the currently claimed method is also suitable for monitoring changes in the cell cycle, and changes associated with cell differentiation and cell death.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in chemistry or related fields are intended to be within the scope of the following claims.

TABLE I
Peak assignments for the MLE-12 Raman
spectrum [1, 5, 6, 14, 26]

| | |
|---|---|
| Peak position | Assignment |

| (cm⁻¹) | DNA | Proteins |
|---|---|---|
| 1659 | | Amide I |
| 1607 | | Phe, Tyr |
| 1578 | G, A | |
| 1487 | G, A | |
| 1460 | | CH def |
| 1449 | | CH def |
| 1422 | A, G | |
| 1375 | T, A, G | |
| 1339 | A | CH def |
| 1305 | A | CH def |
| 1252 | A | Amide III |
| 1240 | T | Amide III |
| 1209 | | Phe, Tyr |
| 1176 | | Tyr, Phe |
| 1155 | Ribose-Phosphate | |
| 1126 | | C—N, C—C str |
| 1094 | Backbone PO₂⁻ sym str | |
| 1032 | | Phe |
| 1004 | | Phe |
| 937 | | Skel. C—C str |
| 900 | Backbone | |
| 853 | | Tyr |
| 833 | Ribose-Phosphate | |
| 830 | | Tyr |
| 789 | C, T, Backbone OPO sym | |
| 751 | T (ring breathing) | |
| 729 | A (ring breathing) | |
| 684 | G (ring breathing) | |
| 669 | T, G | |
| 645 | | Tyr (skeletal) |
| 623 | | Phe (skeletal) |

1. A method of eliciting a Raman signal from a living cell, or a plurality of living cells, said method comprising irradiating the cell with a laser having a wavelength of 785±60 nm.

2. A method according to claim 1 comprising irradiating the cell with a laser having a wavelength of 785±20 nm.

3. A method according to claim 1 wherein the cell is exposed to a total energy of at least about 20 Joules.

4. A method according to claim 1 wherein the cell is exposed to a total energy of at least about 100 Joules.

5. A method according to claim 1 wherein the cell is exposed to a total energy of at least about 200 Joules.

6. A method according to claim 1 wherein the cell is exposed to a total energy of at least 275 Joules.

7. A method according to claim 1 wherein the cell is irradiated at an intensity of 115±50 mW.

8. A method according to claim 1 wherein the cell is irradiated at an intensity of 120±60 mW.

9. A method according to claim 1 wherein the cell is irradiated for a period of up to 40 minutes.

10. A method according to claim 1 wherein the laser is focussed within the cytoplasm of the cell.

11. A method according to claim 1 wherein the laser is focussed within the nucleus of the cell.

12. A method according to claim 1 wherein the laser is focussed within the extracellular matrix.

13. A method according to claim 1 wherein the cell is cultured on a bioinert material.

14. A method according to claim 13 wherein the bioinert material is poly-L-lysine coated fused silica.

15. A method according to claim 1 wherein the cell is cultured on a bioactive scaffold.

16. A method according to claim 1 wherein the cell is cultured on an uncoated bioactive glass or a sol-gel derived gel glass.

17. A method of detecting changes in a living cell or a plurality of living cells, said method comprising the steps of:
(i) eliciting a Raman signal in accordance with claim 1; and
(ii) measuring changes in the Raman signal over a period of time.

18. A method according to claim 17 for detecting changes in one of the cell phenotype, induced by a pharmaceutical agent or a cytotoxic agent, protein levels, DNA or RNA levels, the extracellular matrix, the cell cycle.

19. A method according to claim 17 for monitoring cell growth.

20. (canceled)

21. (canceled)

22. (canceled)

23. (canceled)

24. A method according to claim 17 for detecting the cell cycle of a living cell.

25. (canceled)

26. A method according to claim 17 for detecting the onset of cell death by apoptosis or necrosis.

27. (canceled)

28. (canceled)

29. (canceled)
